(19) United States
(12) Patent Application Publication
(10) Pub. No.: US 2005/0211254 A1
(43) Pub. Date: Sep. 29, 2005

(54) Method and apparatus for forming ear and nose plugs

(76) Inventor: Mark A. Olson, Price, UT (US)

Correspondence Address: MORRISS O'BRYANT COMPAGNI, P.C., 136 SOUTH MAIN STREET, SUITE 700, SALT LAKE CITY, UT, 84101, US

(21) Appl. No.: 10/806,625

(22) Filed: Mar. 23, 2004

(51) Int. Cl. A61F 11/00

(52) U.S. Cl. 128/864

(57) ABSTRACT

The invention includes an ear or nose plug and a method of forming such an ear or nose plugs. The method may include providing silicone rubber base and catalyst materials and mixing about equal parts of the silicone rubber base and catalyst materials to form a homogeneous putty. The method may further include forming a bullet shaped and sized module of the homogeneous putty. The method may further include inserting the module within an ear or nose cavity using enough gentle pressure to allow the module to conform to the ear or nose cavity and leaving enough of the module outside of the cavity to grasp for removal. The method may further include waiting a predetermined amount of time to cure and removing a custom shaped ear or nose plug. Embodiments of an ear or nose plug formed by the above method are also disclosed. The ear or nose plugs are formed in situ and quickly cure according to the methods of the present invention.

### BACKGROUND OF THE INVENTION

1. Field of the Invention: This invention relates generally to ear and nose plugs, and specifically relates to a method and apparatus for forming ear and nose plugs.

2. Description of Related Art: Ear plugs are useful for protecting the human ear from excessive noise and may also prevent water or other liquids and solids from entering the ear canal. Conventional ear plugs may be formed of cotton, cotton wool and cotton wool fibers impregnated with wax-like substances having shapeable properties. These conventional cloth-based ear plugs tend to fall out of the ear easily.

Other conventional ear plugs may be formed of rubber and rubber-like substances that are elastically deformable. Such rubber ear plugs may stay in the ear better than cloth-based ear plugs, but are less comfortable to wear for long periods of time because they are only elastically deformable and are shaped in a round cross-section which may not be the same as the ear canal and are thus not a custom fit.

Still other conventional ear plugs may be formed of a compressible foamed polymer structure that may be compressed by the use of pressure and then introduced into the ear canal in the compressed state. The compressed foam polymer structure slowly expands to fill the ear canal and, thus, form an ear plug that seals the ear canal more comfortably than rubber ear plugs. However, one drawback associated with conventional ear plugs formed of foamable polymers (e.g., soft polyvinyl chloride and polyurethane) is that they often include low molecular softeners, stabilizers or other processing residues that cannot be removed from the finished ear plug that may lead to skin irritations, contact dermatitis and other allergic skin reactions. Additionally, such foamed polymer structures can exert an uncomfortable force on the outer ear canal.

GB2172508A to Rowe discloses a deformable ear plug formed of a material such as silicone that sustains its shape but can be manually changed from one shape to another and can be shaped with an extension for grasping upon removal. The Rowe ear plug does not permanently retain the shape of the ear canal upon removal because of its deformable material properties.

U.S. Pat. No. 4,459,247 to Rothemund discloses a method for producing ear plugs of foamed plastic that retains a final form using polysiloxane materials. However, the Rothemund method requires foaming and cross-linking an extruded, preshaped polysiloxane mixture, followed by tempering at elevated temperatures (at least 140° C.) for an extended period of time (6 hours). The Rothemund tempering process appears unsuitable for in situ formation of a custom fit ear plug.

U.S. Pat. No. 3,782,379 to Lampe discloses a method of forming room temperature vulcanizable silicone rubber ear plugs. The silicone rubber composition according to Lampe includes a high molecular weight liquid diorganopolysiloxane having terminal hydroxyl groups bonded to the silicon atom (up to 60 percent by weight of which may be a low molecular weight liquid diorganopolysiloxane), filler, alkyl silicate as a cross-linking agent and a metallic salt of an organic monocaboxylic acid catalyst. The Lampe ear plugs may be formed in situ and cure enough to be removed within 20-30 minutes from the start of mixing. However, full curing of the Lampe ear plugs may take up to 6 hours.

Thus, there still exists a need in the art for alternative methods of forming ear plugs in situ that cure faster than conventional silicone rubber ear plugs such that the ear plugs permanently retain the shape of the ear canal after curing. Such alternative methods may also find application in nose plugs and for other suitably sized orifices (bodily or otherwise).

### BRIEF SUMMARY OF THE INVENTION

An embodiment of a method of forming ear or nose plugs according to the present invention is disclosed. The method may include providing silicone rubber base and catalyst materials and mixing about equal parts of the silicone rubber base and catalyst materials to form a homogeneous putty. The method may further include forming a ball or bullet shaped and sized module of the homogeneous putty. The method may further include inserting the module within an ear or nose cavity using enough gentle pressure to allow the module to conform to the ear or nose cavity and leaving enough of the module outside of the cavity to grasp for removal. The method may further include waiting a predetermined amount of time to cure and removing a custom shaped ear or nose plug. Embodiments of an ear or nose plug formed by the above method are also disclosed.

Additional features and advantages of the invention will be apparent from the detailed description which follows, taken in conjunction with the accompanying drawings, which together illustrate, by way of example, features of embodiments of the present invention.

### BRIEF DESCRIPTION OF THE SEVERAL VIEWS OF THE DRAWINGS

The following drawing illustrates exemplary embodiments for carrying out the invention. Like reference numerals refer to like parts in different views or embodiments of the present invention in the drawings.

FIG. 1 is a flow chart of an embodiment of a method of forming ear or nose plugs according to the present invention;

FIG. 2A is a partial cross-sectional side view of an ear plug in accordance with the principles of the present invention inserted into an ear canal; and

FIG. 2B is a bottom view of the ear plug illustrated in FIG. 2A.

### DETAILED DESCRIPTION OF THE INVENTION

Embodiments of the present invention include ear and nose plugs suitable for use on humans. While the following embodiments are directed to human applications, the invention can easily be extended to other animals. Additionally, the plugs and methods described herein may be used to form a plug for any similarly sized orifice. Advantages of the ear and nose plugs of the present invention include ease of use and forming, the plugs permanently retain their shape once formed and can be reused numerous times. In addition, because the plugs permanently maintain their shape once removed from an orifice, the plugs are custom fitting to a particular individual. This custom fit provides a tight, yet comfortable fit for the wearer. That is, the plugs do not significantly expand or contract during curing, the plugs retain the shape of the orifice and when inserted provide a substantially water proof seal between the plug and the orifice, and when used in the ear canal significantly dampen sound waves entering the ear canal.

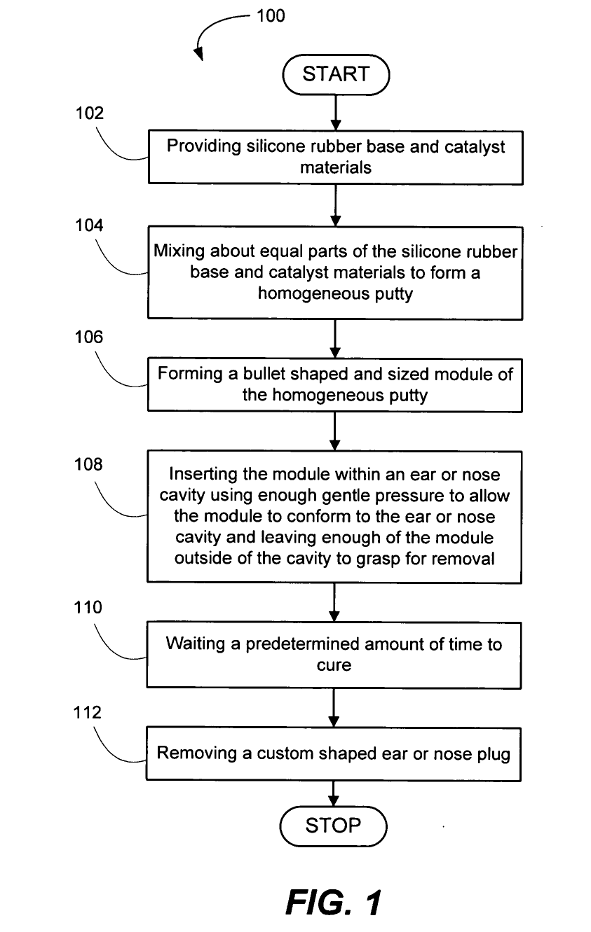

FIG. 1 is a flow chart of an embodiment of a method 100 of forming ear or nose plugs according to the present invention. Method 100 may include providing 102 silicone rubber base and catalyst materials and mixing 104 about equal parts of the silicone rubber base and catalyst materials to form a homogeneous putty. Method 100 may further include forming 106 a ball or bullet shaped and sized module of the homogeneous putty. Method 100 may further include inserting 108 the module within an ear or nose cavity using enough gentle pressure to allow the module to conform to the ear or nose cavity and leaving enough of the module outside of the cavity to grasp for removal. Method 100 may further include waiting 110 a predetermined amount of time to cure and removing 112 a custom shaped ear or nose plug.

There are two types of silicone rubber materials used for making impressions in the dental prosthesis industry that may be identified on the basis of their chemical reactions: condensation and addition types. The presently preferred silicone rubber material for the ear and nose plugs of the present invention are of the addition type. Providing 102 silicone rubber base material may include providing a polysiloxane base having moderately low molecular weight polymer with 3 to 10 silane groups per molecule and filler according to an embodiment of the present invention. Providing 102 silicone rubber catalyst material may include providing a moderately low molecular weight polymer with vinyl terminal groups plus filler and chloroplatinic acid catalyst according to another embodiment of the present invention. Of course other suitable silicone rubber base and catalyst materials having similar properties are also contemplated to be within the scope of the present invention. A source for a silicone rubber base and catalyst material suitable for method 100 is Correct Plus™ vinyl polysiloxane impression material available from Pentron® Clinical Technologies, LLC, P.O. Box 724, Wallingford, Conn. 06492. Other equivalent sources will be readily apparent to one skilled in the art.

Mixing 104 may include kneading by hand to obtain uniformly mixed homogeneous putty according to an embodiment of the present invention. Mixing 104 may include using a spatula to obtain uniformly mixed homogeneous putty according to another embodiment of the present invention. Mixing 104 may also be accomplished by using an auto-mix cartridge that allows dispensing of equal parts base and catalyst simultaneously and then either kneading the equal parts by hand or using a spatula or other suitable instrument according to other embodiments of the present invention. Where the silicone rubber base and catalyst materials are of two different colors, mixing 104 may be complete when the two different colors are uniformly blended and substantially streak free. The mixed silicon rubber material may be of low, medium or high viscosities. High viscosity mixed silicone rubber material is a presently preferred embodiment of the present invention.

It is recommended that latex rubber gloves be avoided when mixing 104 the silicone rubber base and catalyst materials by hand. This is because latex rubber gloves are formed by a vulcanization process using sulfur compounds that can migrate to the surface of the gloves during storage. During mixing of the silicone rubber base and catalyst materials, such sulfur compounds may be incorporated into the homogeneous putty and poison the platinum-containing catalyst, resulting in a retarded or nonexistent polymerization. Washing of the latex gloves with detergent and water just before mixing sometimes minimizes this effect. Additionally, some brands of latex gloves are more effective in interfering with the polymerization or curing during setting than are others. Vinyl gloves do not have such an effect and are thus suitable for use during mixing 104.

Forming 106 a relatively small ball or bullet shaped and sized module of the homogeneous putty for an ear plug may include rolling a ball of between about 0.5 to about 1.5 cubic centimeters of the homogeneous putty between fingers until one end is narrower in diameter than an opposite end according to an embodiment of the present invention. Forming 106 a bullet shaped and sized module of the homogeneous putty for a nose plug may include rolling a ball of between about 2 to about 4 cubic centimeters of the homogeneous putty between fingers until one end is narrower in diameter than an opposite end according to another embodiment of the invention. Of course, the ranges given above are presently preferred embodiments. For ear or nose cavities that are larger or smaller than normal human range, the use of more or less homogeneous putty is contemplated for such applications.

Inserting 108 a module within an ear or nose cavity may be accomplished within about 1 minute to about 3 minutes from beginning of the mixing according to an embodiment of the present invention. Waiting 110 a predetermined amount of time to cure may include waiting at least about 3 to 5 minutes measured from beginning of the mixing according to another embodiment of the present invention. Removing 112 a custom shaped ear or nose plug may include grasping the module outside of the cavity and gently removing the custom shaped ear or nose plug according to yet another embodiment of the present invention.

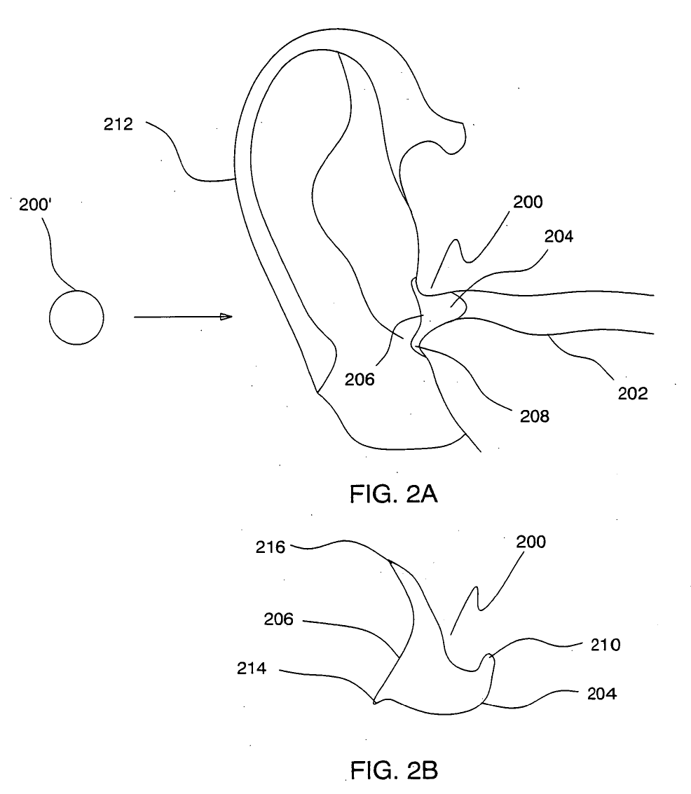

Referring now to FIG. 2A and 2B, an ear plug, generally indicated at 200 is illustrated. The ear plug 200 is formed from a ball 200′ of silicone rubber in uncured form that is inserted at least partially into an ear canal 202 by pressing the ball 200′ at least partially into the ear canal 202. As such, a portion 204 of the plug 200 is fitted within the ear canal 202 and a portion 206 covers the skin at the entrance 208 of the ear canal. Once the plug 200 is cured, the plug will permanently retain the shape of the orifice from which it was formed. As shown in FIG. 4B, which is a bottom view of the ear plug 200, the portion 204 of the ear plug 200 that fits within the ear canal forms a hook-like portion 210 that fits around the ear canal extending toward the back of the ear 212 (see FIG. 4A). This hook-like portion 210 secures the ear plug within the ear canal 202. Thus, when forming the ball 200′, the ball 200′ should be of sufficient size so as to allow formation of the hook-like portion when the ball 200′ is pressed into the ear canal 202. Because the ear plug 200, however, remains flexible after curing, the hook portion 210 can flex to allow easy and painless removal of the ear plug 200 from the ear canal 202 as well as easy and painless reinsertion of the ear plug 200 as desired by a user. To remove the ear plug 200 from the ear canal, the edges 214 and 216 of the outer portion 206 of the ear plug 200 can be grasped between the thumb and forefinger and pulled from the ear canal 202.

According to another embodiment of the present invention a polyvinyldimethylsiloxane or polyvinylsiloxane ear or nose plug may be formed by embodiments of the method 100 described above.

Ear plugs in accordance with the present invention thus have several advantages over the prior art. They are self fabricating and can be adapted to any size wearer from adult to child. They are custom fit to each individual and thus reside comfortably with the ear canal with little, if any, pressure in the ear canal. They are easy formed by simply mixing the silicone rubber with a catalyst to cure the silicone rubber into a pliable material that will permanently hold its molded shape. While allowing plenty of time to form the ear plug, the materials used in accordance with the present invention have relatively quick cure times that can be fully usable within a few minutes. During the curing process, there is little if any increase or decrease in the temperature of the material that may otherwise cause discomfort during curing. Also, because of the relatively high viscosity of the pre-cured putty, the putty will not flow deep into the ear canal that could otherwise cause discomfort when inserting or removing the ear plug. The ear plugs provide thermal protection to the ear canal from cold outside temperatures as well as excellent hearing protection. The outside of the ear plugs are also relatively flush with the ear when inserted so that they are not easily dislodged from external contact. The ear plugs have good retention within the ear canal and can be washed with soap and water and reused over and over again. Because the ear plugs do not put any significant pressure on the ear canal, they are comfortable to sleep with and can be worn without discomfort for extended periods. Moreover, children can wear them without discomfort because they can be individually formed to the precise size of a child's ear canal. The ear plugs are also relatively inexpensive to manufacture, strong and resilient when formed and are formed from inert materials that will not cause allergic reactions with surrounding tissues. The ear plugs according to the present invention can be used while for hearing protection or sound deadening while sleeping, working, hunting, airplane travel and are also useful for thermal protection form cold, to prevent water from entering the ear canal, air equalization while flying and wind protection.

While the foregoing advantages of the present invention are manifested in the illustrated embodiments of the invention, a variety of changes can be made to the configuration, design and construction of the invention to achieve those advantages. For example, the above embodiments are directed to ear and nose plugs for humans. Other applications of the disclosed ear and nose plugs include mammals and other animals where there is a need to form a plug in a body cavity such as an ear or nose. Hence, reference herein to specific details of the structure and function of the present invention is by way of example only and not by way of limitation.

1. A method of forming ear or nose plugs, comprising:
providing silicone rubber base and catalyst materials;
mixing about equal parts of the silicone rubber base and catalyst materials to form a homogeneous putty;
forming a module of the homogeneous putty;
inserting the module within an ear or nose cavity using enough gentle pressure to allow the module to conform to the ear or nose cavity and leaving enough of the module outside of the cavity to grasp for removal;
waiting a predetermined amount of time to cure; and
removing a custom shaped ear or nose plug.

2. The method according to claim 1, wherein providing silicone rubber base material comprises providing a polysiloxane base having moderately low molecular weight polymer with 3 to 10 silane groups per molecule and filler.

3. The method according to claim 1, wherein providing silicone rubber catalyst material comprises providing a moderately low molecular weight polymer with vinyl terminal groups plus filler and chloroplatinic acid catalyst.

4. The method according to claim 1, wherein mixing comprises kneading by hand to obtain a uniformly mixed homogeneous putty.

5. The method according to claim 1, wherein mixing comprises using a spatula to obtain a uniformly mixed homogeneous putty.

6. The method according to claim 1, wherein forming a bullet shaped and sized module of the homogeneous putty for an ear plug comprises rolling a ball of between about 0.5 to about 1.5 cubic centimeters of the homogeneous putty between fingers until one end is narrower in diameter than an opposite end.

7. The method according to claim 1, further comprising forming the module into a bullet shaped and sized module.

8. The method according to claim 7, wherein forming a bullet shaped and sized module of the homogeneous putty for a nose plug comprises rolling a ball of between about 2 to about 4 cubic centimeters of the homogeneous putty between fingers until one end is narrower in diameter than an opposite end.

9. The method according to claim 1, wherein inserting the module is performed within about 1 minute to about 3 minutes from beginning of the mixing.

10. The method according to claim 1, wherein waiting a predetermined amount of time to cure comprises waiting at least about 3 to 5 minutes measured from beginning of the mixing.

11. The method according to claim 1, wherein removing a custom shaped ear or nose plug comprises grasping the module outside of the cavity and gently removing the custom shaped ear or nose plug.

12. A polyvinyldimethylsiloxane or polyvinylsiloxane ear or nose plug formed by the process comprising:
providing silicone rubber base and catalyst materials;
mixing about equal parts of the silicone rubber base and catalyst materials to form a homogeneous putty;
forming a module of the homogeneous putty;
inserting the module within an ear or nose cavity using enough gentle pressure to allow the module to conform to the ear or nose cavity and leaving enough of the module outside of the cavity to grasp for removal;
waiting a predetermined amount of time to cure; and
removing a custom shaped ear or nose plug.

13. The method according to claim 12, wherein providing silicone rubber base material comprises providing a polysiloxane base having moderately low molecular weight polymer with 3 to 10 silane groups per molecule and filler.

14. The method according to claim 12, wherein providing silicone rubber catalyst material comprises providing a moderately low molecular weight polymer with vinyl terminal groups plus filler and chloroplatinic acid catalyst.

15. The method according to claim 12, wherein mixing comprises kneading by hand to obtain a uniformly mixed homogeneous putty.

16. The method according to claim 12, wherein mixing comprises using a spatula to obtain a uniformly mixed homogeneous putty.

17. The method according to claim 12, wherein forming a module comprises forming a bullet shaped and sized module.

18. The method according to claim 17, wherein forming a bullet shaped and sized module of the homogeneous putty for an ear plug comprises rolling a ball of between about 0.5 to about 1.5 cubic centimeters of the homogeneous putty between fingers until one end is narrower in diameter than an opposite end.

19. The method according to claim 17, wherein forming a bullet shaped and sized module of the homogeneous putty for a nose plug comprises rolling a ball of between about 2 to about 4 cubic centimeters of the homogeneous putty between fingers until one end is narrower in diameter than an opposite end.

20. The method according to claim 12, wherein inserting the module is performed within about 1 minute to about 3 minutes from beginning of the mixing.

21. The method according to claim 12, wherein waiting a predetermined amount of time to cure comprises waiting at least about 3 to 5 minutes measured from beginning of the mixing.

22. The method according to claim 12, wherein removing a custom shaped ear or nose plug comprises grasping the module outside of the cavity and gently removing the custom shaped ear or nose plug.

23. A silicone rubber ear plug, comprising,
a first portion for extending into an ear canal and having a hook portion at an end thereof for retaining the ear plug within the ear canal; and
a second portion extending over the skin forming the entrance of the ear canal for grasping the ear plug for removal from the ear canal.

24. The ear plug of claim 23, wherein the ear plug is comprised of at least one of polyvinyldimethylsiloxane or polyvinylsiloxane.

25. The ear plug of claim 23, wherein the ear plug is permanently formed in the shape of a portion of an ear canal.

26. The ear plug of claim 25, wherein the ear plug is formed from a flexible and resilient material.
